(19) United States
(12) Patent Application Publication
(10) Pub. No.: US 2026/0213011 A1
(43) Pub. Date: Jul. 23, 2026

(54) SYSTEM AND ARCHITECTURE FOR PROCESSING ANATOMICAL THREE-DIMENSIONAL DATA

(71) Applicant: XRPro, LLC, Boulder, CO (US)

(72) Inventors: Anton Aleksandrovich Tokar, Longmont, CO (US); Ravi Vibhakar Shah, Boulder, CO (US); Dmitrii Aleksandrovich Gladyshev, Longmont, CO (US); Michael Trevor O'Connell, Louisville, CO (US); Paulo E. Xavier da Silveira, Boulder, CO (US); Jonathan Dana Edwards, San Jose, CA (US)

(21) Appl. No.: 19/142,715

(22) PCT Filed: Jan. 25, 2024
(86) PCT No.: PCT/US2024/012863

§ 371 Date: Jun. 24, 2025

Related U.S. Application Data
(60) Provisional application No. 63/481,685, filed on Jan. 26, 2023.

(51) Int. Cl. G16H 50/20; A61B 34/10; G16H 10/60

(52) U.S. Cl. CPC G16H 50/20; A61B 34/10; G16H 10/60; A61B 2034/105

(57) ABSTRACT

A system for determining features and metrics associated with three-dimensional data of a feature of a patient in substantially real-time. In some examples, the system may include user equipment for capturing three-dimensional scans of the patient and a cloud-based system for processing the three-dimensional data. The system may provide visualization of the three-dimensional data to the users concurrently with the scanning of the feature of the patient.

### CROSS-REFERENCE TO RELATED APPLICATION(S)

This application is a U.S. national stage application under 35 USC § 371 of International Application No. PCT/US24/12863 filed on Jan. 25, 2024 and entitled “SYSTEM AND ARCHITECTURE FOR PROCESSING ANATOMICAL THREE-DIMENSIONAL DATA,” which claims priority to U.S. Provisional Application No. 63/481,685 filed on Jan. 26, 2023 and entitled “METHODS AND SYSTEMS FOR PROCESSING ANATOMICAL 3D DATA USING SERVERS ACCESSED OVER THE INTERNET,” the entire contents of which are incorporated herein by reference.

### BACKGROUND

Specialized healthcare systems that utilize image data for assisting in diagnosing specific symptoms and conditions are becoming more and more common. However, these conventional specialized healthcare systems are expensive to design, build, and implement while also having limited usability to a specific symptom and condition. Accordingly, today, healthcare equipment manufacturers often implement and healthcare providers often purchase a large number of these customized systems. Additionally, these conventional specialized healthcare systems are often required to be present at the location of treatment or diagnostics resulting in limited scope of use.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is an example block diagram of an architecture for a cloud-based anatomical data processing system according to some implementations.

FIG. 2 is another example block diagram of an architecture for a cloud-based anatomical data processing system according to some implementations.

FIG. 3 is another example block diagram of an architecture for a cloud-based anatomical data processing system according to some implementations.

FIG. 4 is another example block diagram of an architecture for a cloud-based anatomical data processing system according to some implementations.

FIG. 5 is another example block diagram of an architecture for a cloud-based anatomical data processing system according to some implementations.

FIG. 6 is an example process flow diagram associated with the cloud-based anatomical data processing system of FIGS. 1-5 described according to some implementations.

FIG. 7 is an example cloud-based anatomical data processing system that may implement the techniques described herein according to some implementations.

FIG. 8 is an example user equipment that may implement the techniques described herein according to some implementations.

FIG. 9 is an example pictorial diagram that shows three-dimensional data of a foot of a patient according to some implementations.

FIG. 10 is another example pictorial diagram that shows three-dimensional data of a spine of a patient according to some implementations.

FIG. 11 is another example pictorial diagram that shows 3D data of a face of a patient according to some implementations.

### DETAILED DESCRIPTION

Discussed herein are systems and architecture for the processing of three-dimensional (3D) data (such as 3D medical data, healthcare data, image data, and/or the like) via cloud-based services, systems, and/or processing resources. In some examples, the cloud-based services may include, among other elements, one or more servers in communication with one or more user equipment or devices over one or more networks. In some implementations, the 3D data may include numeric representations of anatomy of patients or users, such as three-dimensional scans of body parts, portions of skin, organs (internal or external), and the like. The 3D data may also include different types of data, such as thermal data, red-green-blue data, depth data, infrared data, Magnetic resonance imaging (MRI) data, light detection and ranging (LIDAR) data, and the like. In some cases, the 3D data may also include additional data related to the image data such as sensor data including one or more of temperature, oxygenation, bacterial load, electrical potential, dielectric impedance, electrocdiogam (EKG), photplethysmograph (PPG), heart rate, heart rate variance (HRV), and the like. In some cases, meta-data may be associated with the 3D data. For instance, the meta data may include patient information (e.g., identifiers, demographic information, name, age, gender, weight, body part dimensions, such as extracted from the 3D data by the capture device, birth date, medical history, family data, and the like), 3D scan or scanning device information (e.g., device identifier, sensor type, serial number, firmware or software version, scan date, time, and/or the like).

The system discussed herein may receive the 3D data as well as any associated meta data from the user equipment (such as the scanning device) via one or more networks. The system may process the 3D data, as discussed below, to assist a physicians, clinicians, or other health professional with diagnostics, treatment or therapy recommendations, and the like via, for instance, phenotype or individual observable trait determination. For example, the system may enable determination of specific anatomy of specific body parts of individual patients, at multiple and specific points in time, automated identification of anatomical landmarks (e.g., computer definable features), determination of automated measurements of individual patients (such as anthropometric measurements e.g., body part lengths, girths, widths, and the like).

In some implementations, the system, discussed herein, may be configured to provide cloud-based processing of the 3D data to present the advantage of providing users (e.g., healthcare providers) with access to larger, faster, and more efficient and varied computing resources than is available via conventional in-clinic or in-house devices. The advantages of cloud-based processing are considerable when compared to resources available to clinicians in small clinics or remote geographical areas. In some case, as discussed herein, cloud-based processing may consist of multiple servers, available full-time and on demand, making their services substantially ubiquitous, constantly available, on demand and easily accessible (e.g., additional servers may be quickly activated in times of peak demand). Also, servers may consist of multiple computers in large service centers, using different operating systems, benefiting from lower-cost centralized utilities and services, and from expandable infrastructure, such as multiple parallel central processing units (CPUs), graphic processing units (GPUs), arithmetic logic units (ALUs), tensor processing units (TPUs) and quantum processing units (QPUs), to name a few. In general, cloud-servers may also be referred to as backend-servers or simply backend.

In addition, the cloud-based system discussed herein may include data pre-processing, use of one or more machine learning models that are trained on 3D data associated with individuals having various conditions, symptoms, states of health, age, genders, cultural backgrounds, and the like. For example, the one or more machine learning models may be trained to segment the 3D data, classify the 3D data, perform feature detection (such as identifying body parts, landmarks, dimensions, and the like) from the 3D data. The one or more machine learning models may also be trained to assist in diagnosing conditions and symptoms with respect to various body parts and individuals having a wide variety of features (such as those classified and identified by one or more other machine learning models), determining health status, recommending patient specific treatments or therapies and the like. In this manner, a health care professional may upload 3D data via a user equipment to the cloud-based system and receive in response indications of body parts or conditions that may require further evaluation, user specific anthropometric measurements to assist with diagnostics or evaluations, flagged or identified potential conditions, symptoms as well as suggested treatments or therapies. In some cases, the cloud-based system may provide instructions to perform additional scans and/or capture additional 3D data associated with a specific user to enhance any recommendations or features identified. In one specific example, the cloud-based system may also return one or more additional inquiries for the healthcare professional and/or the patient, such as questions related to an accident, a particular body part, history of a body part or feature detected, and the like to further assist the healthcare professionals in diagnostics and evaluation of the patient.

In one specific example, the user equipment may be utilized by a healthcare professional to scan a patient to capture the 3D data that is provided to the cloud-based system. For example, the user (e.g., healthcare professional) may utilize the user equipment to enter patient data and initiate a scanning session of the patient to capture both the meta data (e.g., the patient data) and the 3D data, such as via a software development kit (SDK) or downloadable application. Both the meta data and the 3D data associated with the patient may then be provided via one or more networks to the cloud-based system. In some cases, the user may label the 3D data, such as to identify the body part or patient being scanned. During the scan and on the user equipment, the SDK or downloadable application may provide a real-time visualization of the 3D data, such as on a display of the user equipment, to assist the user in capturing the visual data.

In some cases, the real-time visualization may include indictors to the user of a progress or completion of the scan (e.g., the system may change a visual characteristic of the 3D data on the display as the scan is captured or meets or exceeds various thresholds). As another example, the real-time visualization may include a progress bar or visual indication or areas that remain unscanned or that insufficient data was captured to accurately process by the cloud-based system. In some case, the SDK or downloadable application may perform pre-processing on the 3D data to determine if sufficient data was captured for use by the cloud-based system (e.g., gaps or holes in the 3D scan, proper lighting during capture, depth data was properly captured, or the like), such as via a substantially real-time tracking or simulation location and mapping (SLAM) function. In some cases, the user equipment may associate other data (e.g., meta data) with the 3D data, such as IMU data, room and/or body temperature data, time, date, physical location, length of time associated with the scan, user operating or logged into the equipment, and the like.

The SDK or downloadable application may also provide a local version of the 3D data and/or meta data prior to sending to the cloud-based system. For instance, the local version may include a Truncated Signed Differences Function (TSDF) or other voxel representation of a portion of the patient's body, such as for example, generated via a mapping process. The SDK or downloadable application may also generate one or more meshes associated with the scan from the 3D data or format the mesh and/or 3D data into one or more types of files for output, such as encrypting and encoding the 3D data and meta data prior to transmitting to the cloud-based service.

In this example, once the cloud-based system has received the 3D data and any associated meta data, the cloud-based system may decrypt and decode the data. The cloud-based system may then generate keys and re-encrypt (e.g., via a different encryption than the SDK or downloadable application applied) any patient-identifiable data with, for instance, a government approved encryption method based on various patient and server physical locations. The cloud-based system may then provide the encrypted patient data to various systems such as a datastore associated with the health care provider associated with the scan. The cloud-based system may also extract the non-patient data (e.g., data not usable to identify the patient) as anonymized data that may be searchable, used for processing (such as machine learning model training), and the like. In some cases, the data may be partitioned into a training dataset, a testing dataset and a validation dataset that may be used to train, test, and validate the operations of the machine learning models and/or networks.

The system may also utilize the 3D data, the pre-processed data (e.g., the mesh, TSDF, and the like), and the associated meta data to perform various diagnostics assistance features. For instance, the cloud-based system may determine, classify, or identify body-parts, landmarks, anthropometric metrics or features, potential conditions or symptoms, recommend treatments, or the like. In various instances, the additional processes may be performed on-demand and/or at the request of the user (e.g., the healthcare professional). For instance, the user may select the processing to be performed by the cloud-based system via the SDK or downloadable application on the user equipment prior to or in conjunction with transmitting the 3D data to the cloud-based system. In this manner, the user may limit the processing performed by the cloud-based system based at least in part on the services being provided to the patient.

In some cases, the cloud-based system may include a priority based queue. For example, based on the type of processing, class of the user, health or condition of the patient, and the like the data processing may be prioritized. In this manner, the cloud-based system may provide urgent processing for life threatening, serious, or triage (e.g., emergency room) related treatments or conditions. In other examples, the cloud-based system may provide prioritized processing based on timestamp associated with when 3D data was uploaded, status of a developer, status of the user, a premium payment, size of the scan, availability of processing resources, and the like.

The cloud-based system may provide any generated data, recommendations, and the like back to the user equipment for review by the healthcare professional, to various third-party systems (such as an insurance provider system, health specialist system, research system, or the like), and/or to a system or portal associated with the patient. For example, the anatomical data processing system may be configured to complete or fill forms (e.g., health forms, government forms, insurance forms, and the like) and to provide end-users (e.g., patients, healthcare provides, and the like) copies of the forms submitted and/or competed. In this manner, the system may enable sending or transmission of forms or documents to medical insurance providers, government entities, patients, other healthcare professionals and the like.

In one specific example, the user may be the patient and the scan may be captured via a user device (such as a smartphone, tablet, or the like) associated with the patient via a self-scan. In this manner, the system may facilitate remote medicine or telemedicine, such as when the patient is assisted by a remote healthcare professional. For example, a patient may capture scans of wounds, ulcers, moles, scabs, and other skin conditions, using their personal device and camera. These scans may be provided to the cloud-based system and/or to remote healthcare providers. For instance, a patient suffering from an infection while on a remote hike may utilize their user device and the cloud-based system to receive recommended actions or to inform a healthcare professional who can provide recommended actions or dispatch medical evacuation services, or the like.

In some examples, the cloud-based anatomical data processing system may provide features for use or accessible via an application programming interface (API) that may be accessed as part of a third-party solution. For instance, the system may provide a government compliant datastore associated with the personal health information or data including the 3D data and the associated meta data. The system may also provide processing for and associated with substantially real-time and/or real-time streamed data acquisition, 3D data reconstruction, such as an “on the fly” process, autocalibration of the 3D data and/or the sensor capturing the data, generation of a water-tight or fully enclosed (e.g., without exterior openings or holes) model, generating meshes of the 3D data at various resolutions or quality, providing adaptive re-meshing features (e.g., a TSDF may be sued to produce a mesh, such as via a marching cubes technique, but with an increased resolution in some areas, such as areas with high curvature, or that are desirable for clinical application), providing photogrammetry reconstruction features, and the like. In some cases, the system may generate or construct meshes using multi-sensor fusion, wherein the final reconstructed object or body part has higher resolution and/or accuracy than that obtain by a single sensor, aligning the mesh, and the like.

In some examples, the cloud-based anatomical data processing system may provide features for editing and/or measuring the resulting 3D model or anatomical features (such as body parts). The system may also assist with finding landmarks on surfaces or within the 3D model and/or generating bounding boxes or regions associated with various anatomical features. The system may also compare metrics or measurements between thresholds, prior scans or models (e.g., of the individual patient), determine trends with respect to the anatomical features or models, and the like. Using the 3D models the system may also generate diagnostic metrics, identify conditions or symptoms, generate treatment or therapy recommendations, and the like. For example, the system may diagnose medical conditions, such as lymphodema, based at least in part on a comparison of measurements of a perimeter of the convex hull of limbs and known medial baseline values or thresholds determined based at least in part on, for instance, a patient's age, gender and BMI (body mass index). In some cases, the diagnostics or recommendations may be provided to a healthcare professional, patient, or representative of the patient using one or more alerts.

In this manner, the cloud-based anatomical data processing system may allow third-party developers or equipment manufacturers to access features via, for instance, an application programming interface (API) resources for processing of health related 3D data, as discussed herein, without designing and implementing custom per symptom or per condition equipment. For instance, a developer may utilize customized hardware designed to, for example, diagnose a specific condition and include one or more sensors for capturing 3D health or anatomical data design together with the cloud-based anatomical data processing system to provide a lower cost solution at a reduced development cycle or time. As one example, the developer may access the cloud-based system utilizing API calls to identify anatomical features, anatomical relations, anatomical measurements, or the like associated with the specific condition. The

In addition to the API and cloud-based processing, the system and design architecture, discussed herein, may include a downloadable application or SDK that may operate on various types of medical or user equipment. The SDK may provide assistance for a device or sensor system to collect patient data (e.g., the meta data associated with the 3D data or scan), such as via a graphical user interface (GUI). The SDK may also include features for encryption, pre-encryption, transmission, storage and the like in a manner that meets or exceeds government regulations. In one specific example, the SDK may provide a GUI for visualizing the 3D data or to assist a user in capturing of the 3D data, as discussed herein.

In various examples, the machine learning models and/or networks may be trained. For example, the 3D data and/or the meta data may be partitioned into a training dataset, a testing dataset and a validation dataset that may be used to train, test, and validate the operations of the machine learning models and/or networks. In some cases, data may overlap into the training dataset, the testing dataset, and/or the validation dataset, such that some data is present in two or more of the datasets. As discussed herein, the training dataset, the testing dataset and the validation dataset may include portions of the 3D data and associated meta data aggregated over a plurality of users with varying demographics (age, gender, cultural background, and the like), physical locations, aliments, conditions, symptoms, health levels, and the like. The training dataset, the testing dataset and the validation dataset may also include image data, thermal data, atomical data (with and/or without segmentation, classification, feature extraction and identification, and the like), meshes, surfaces, TSDF data, DICOM data (e.g., from MRI and computed tomography) and the like.

In one example, the training dataset may be pre-processed prior to use in training one or more machine learning models. For example, the pre-processing may include generation of sub-sampled meshes from full-resolution meshes or other 3D data, alignment of body parts (e.g., hands, heads, feet, arms, legs, chest, lungs, heart, eye, hair, chin, knees, and the like) to align them with respect to a plane or surface of the 3D data, a mesh associated with the 3D data, or the like. In some cases, the pre-processing may also include the formation of inferences based at least in part on the pre-trained models, such as models available in the public healthcare domain. In some cases, the system may then train the one or more machine learning models, using pre-defined datasets, but also including portions of or all of the data in the training dataset, thereby providing an ability to trade off computational power and computational data when needed.

As described herein, the machine learning models may be generated using various machine learning techniques. For example, the models may be generated using one or more neural network(s). A neural network may be a biologically inspired algorithm or technique which passes input data (e.g., image and sensor data captured by the user equipment or devices) through a series of connected layers to produce an output or learned inference. Each layer in a neural network can also comprise another neural network or can comprise any number of layers (whether convolutional or not). As can be understood in the context of this disclosure, a neural network can utilize machine learning, which can refer to a broad class of such techniques in which an output is generated based on learned parameters.

As an illustrative example, one or more neural network(s) may generate any number of learned inferences or heads from the captured sensor and/or image data. In some cases, the neural network may be a trained network architecture that is end-to-end. In one example, the machine learning models may include segmenting and/or classifying extracted deep convolutional features of the sensor and/or image data into semantic data. In some cases, appropriate truth outputs of the model in the form of semantic per-pixel classifications (e.g., vehicle identifier, container identifier, driver identifier, and the like).

Although discussed in the context of neural networks, any type of machine learning can be used consistent with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID 3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like. In some cases, the system may also apply Gaussian blurs, Bayes Functions, color analyzing or processing techniques and/or a combination thereof.

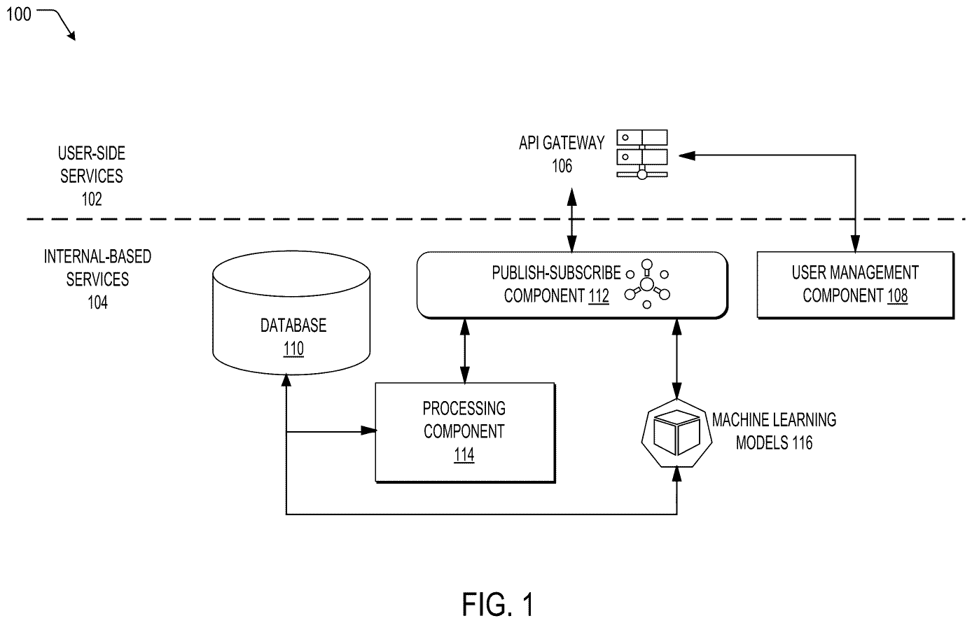

FIG. 1 is an example block diagram of an architecture for a cloud-based anatomical data processing system 100 according to some implementations. In the current example, the system 100 may include a cloud-based service having a user-side service 102 and an internal-based service 104. The user-side service 102 may include the API gateway 106. The API gateway 106 may be configured to receive API calls from user equipment (e.g., health professional equipment, user or patient devices, and the like) via a web-based application, SDK, or downloadable application hosted by the user equipment. In some cases, the API gateway 106 may process or handle user requests, authentications, authorizations, account balance operations, datastore or database access requests, storage of 3D data, meshes, and the like (e.g., non-patient identifiable data), and the like. For example, the API gateway 106 may process and forward authorization or authentication data to a user management component 108 of the internal-based service 104.

In the current example, in addition to the user management component 108, the internal-based service 104 also includes a database 110 accessible by the API gateway 104 and a publish-subscribe component 112 in communication with the API gateway 104. The database 110 may be configured to store the 3D data and the associated meta data in a manner accessible by the API gateway 104. The publish-subscribe component 112 may be configured to set quotas and rate limits for access requests by different users, prioritize incoming tasks and processes, such as discussed above, improve scalability, control access and use of additional components (e.g., as illustrated processing components 114, machine learning models and/or networks 116, and the like), maintain data retention policies for the database 110, and the like.

The processing components 114 (such as a mesh processing component) may be configured in response to receiving a control input from the publish-subscribe component 112 to generate one or more meshes or models of the 3D data of a particular patient stored or maintained in the database 110. For example, based at least in part on the meta data associated with the particular patient, the publish-subscribe component 112 may generate a control input indicting types of meshes, number of meshes, quality of meshes, or the like to be generated by the processing components 114. Once received, the processing components 114 may access the 3D data associated with the particular patient and generate one or more meshes based at least in part on the control input.

Similar to the processing components 114, the one or more machine learning models 116 may be configured to receive a control input from the publish-subscribe component 112 together with data from the datastore, such as the 3D data and/or meta data of the particular patient. In this manner, the control input, the 3D data of the particular patient, and/or the meta data associated with the particular patient may form the input to the machine learning models. The machine learning models 116 may then in response provide and/or store in the database 110 various outputs such as segmented 3D data, condition and/or symptom diagnostics, anatomical features, measurements, and/or metrics, recommended treatments or therapies, recommended further investigational or diagnostic operations for a medical health professional, changes and/or deltas in anatomical features, health based alerts, and the like.

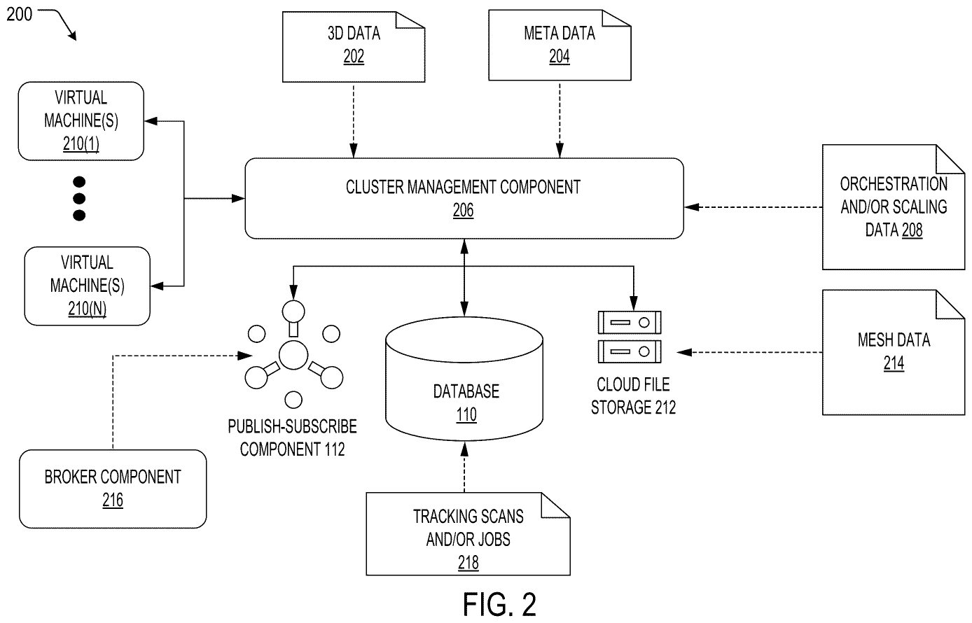

FIG. 2 is another example block diagram of an architecture for a cloud-based anatomical data processing system 200 according to some implementations. In the current example, the 3D data and any associated meta data 204 associated with each scan being processed by the cloud-based anatomical data processing system 200 may be received at a cluster management component 206. The cluster management component 206 may also receive an orchestration and/or scaling data 208 associated with the 3D data 202 for a particular scan. The cluster management component 206 may also be configured to manage one or more virtual machines 210(1)-(N). For example, the cluster management system 206 may cause the virtual machine 210(1) to process the 3D data 202 associated with a particular scan based on the orchestration and/or scaling data 210 and a selected process received from an application or SDK associated with user equipment generating the scan, as discussed above.

In the current example, the cluster management component 206 may also be coupled to the publish-subscribe component 112 and database 110, discussed above with respect to FIG. 1 and a cloud file storage 212. In the current example, the cloud file storage 212 may receive mesh data 214 (and/or other 3D data either in original forms, other formats, or after processing processed) and other non-patient data from the user equipment and store the non-patient data as a persistent record. As illustrated, the publish-subscribe component 112 component may be further configured to receive messages (e.g., internal messages, control signals, communications, and the like between subsystems of the cloud-based anatomical data processing system 200) from a message broker component 216.

In the current example, the database 110 may receive tracking data (such as SLAM data) and/or job data 218 from the user equipment or another component of the system 200. For instance, the jobs data may include health professional requests (e.g., desired measurements, validations, recommendations, and the like).

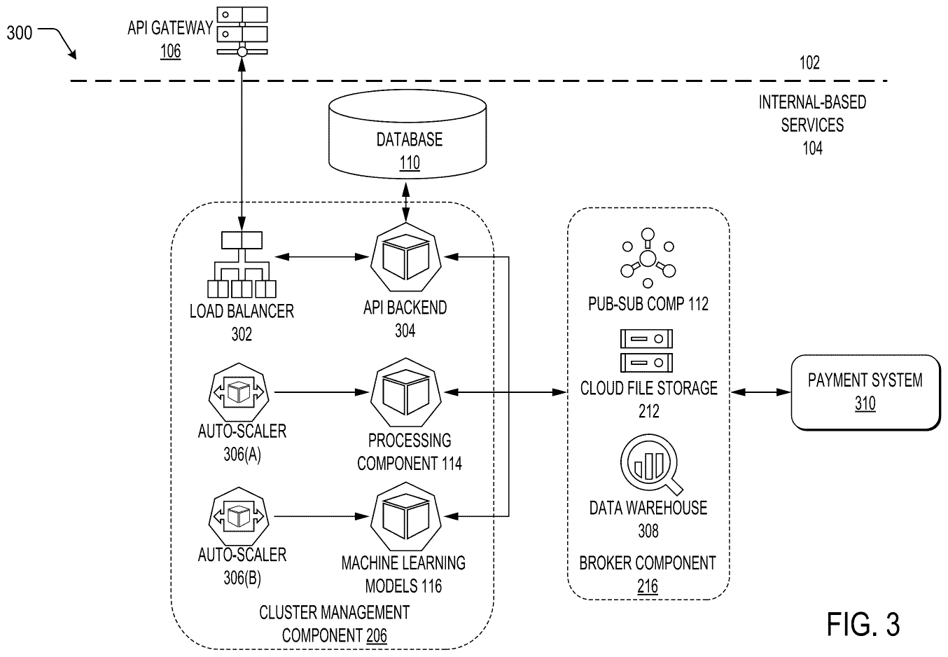

FIG. 3 is another example block diagram of an architecture for a cloud-based anatomical data processing system 300 according to some implementations. In the current example, the system 300 may include a cloud-based service having a user-side service 102 and an internal-based service 104, as discussed above. The user-side service 102 may include the API gateway 106. The API gateway 106 may be configured to receive API calls from user equipment (e.g., health professional equipment, user or patient devices, and the like) via a web-based application, SDK, or downloadable application hosted by the user equipment. In some cases, the API gateway 106 may process or handle user requests, authentications, authorizations, account balance operations, datastore or database access requests, storage of 3D data, meshes, and the like (e.g., non-patient identifiable data), and the like.

Similar to the example of FIG. 2, in this implementation, the API gateway 106 may be in communication with the cluster management component 206. In the current example, the cluster management component 206 may include a load balancer 302, a API backend 304, one or more auto-scalers, such as auto-scalers 306(A) and 306(B), a processing component 114, one or more machine learning models 116, and the like.

As discussed above, the cluster management component 206 may be coupled to the database 110 to store final scan data, tracking data, job data, and the like. In some cases, the database 110 may be implemented as a firewalled system or multiple databases, including at least one portion or database that is government compliant to store personalized health data for a jurisdiction associated with a patient associated with the 3D data. In this manner, the database 110 may be configured to segregate and separately store patient identifiable data (e.g., portions of the meta data) and non-patient identifiable data (e.g., the scan data and the like).

In the current example, the load balancer 302 and the API backend 304 may be configured to manage system tasks, database accesses, and the like. The auto-scalers 306(A) and 306(B) may each be respectively responsible for managing the operations performed by the processing component 114 and the machine learning models 116. As discussed herein, the processing components 114 may be configured to perform heuristic or process based operations on the 3D data to generate outputs, such as metrics, measurements, landmark detections, and the like, while the machine learning models 116 may be configured to perform diagnostics, identify symptoms or conditions, generate recommended treatments or therapies (e.g., either known or customized or personalized for the patient associated with each 3D data), and the like. In some cases, the machine learning models 116 may be trained using historical patient data and 3D image data of patients with and without various types of symptoms, conditions, ailments, and the like. The machine learning models 116 may also be trained on image data including 3D data of various portions of patient's bodies having varying dimensions, colorations, landmarks, and the like. In some cases, the training data may also include meta data associated with each 3D scan. For instance, the training data may include cultural data, demographic data, location data, environmental data, and the like for each training scan. The training data may further include DICOM data, such as MRI and CT scan 3D data representative of relevant medical conditions, such as cancer, lymphedema and pes planus (i.e., flat foot).

In the current implementation, the publish-subscription component 112, the cloud file storage 212 (for storing the 3D data, image data, mesh data, and the like) are incorporated into the broker component 216. The broker component 216 may also include a data warehouse 308 for storing business data and events, such as transactional data received from a payment system 310.

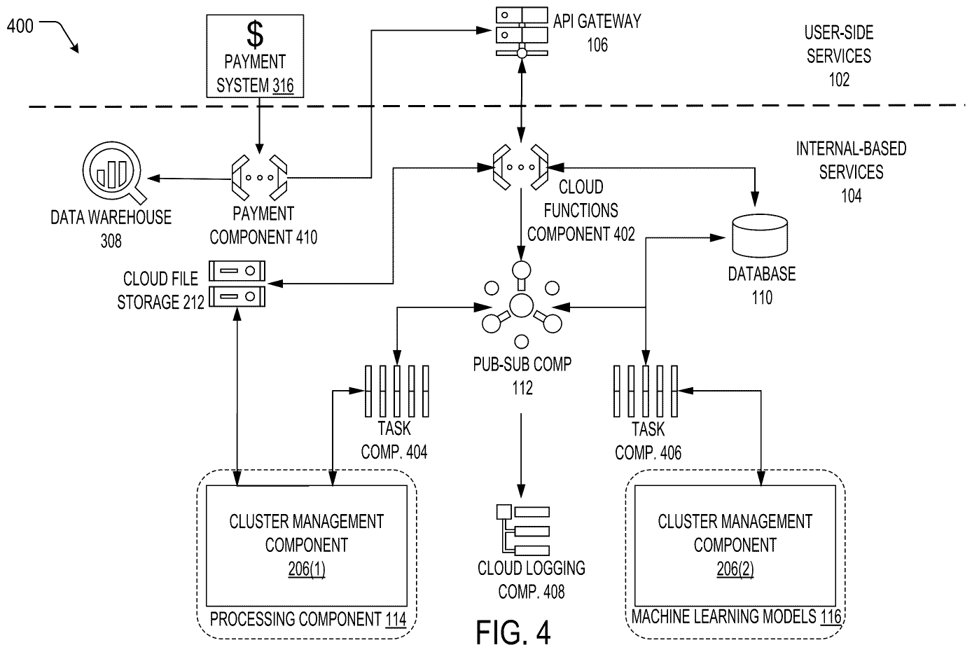

FIG. 4 is another example block diagram of an architecture for a cloud-based anatomical data processing system 400 according to some implementations. In the current example, the API gateway 106 may be in communication with a cloud-function component 402 to process data received from the API gateway 106 and output to the API gateway 106. The cloud-function component 402 may then be coupled to the publish-subscribe component 112, the database 110, and the cloud file storage 212, discussed above with respect to FIGS. 1 and 2. The publish-subscribe component 112 may be further coupled to a task component 404 associated with a first cluster management component 206(1) and a task component 406 associated with a second cluster management component 206(2). Each of the cluster management components 206(1) and 206(2) are, respectively, associated with the operations of the processing component 114 and the machine learning models 116. Accordingly, in the current implementation, the processing component 114 is associated with the task component 404 and the cluster management component 206(1) and the machine learning models 116 are associated with the task component 406 and the cluster management component 206(2). In this manner, the operations, scheduling, and/or processing associated with determining metrics, measurement, landmarks and the like may be separated by the system 400 from the operations, scheduling, and/or processing associated with diagnostics, identifying symptoms and conditions, and generating recommended or patient customized therapy and/or treatments.

The publish-subscribe component 112 may also be coupled to a cloud logging component 408. The cloud logging component 408 may be configured to record events, jobs, tasks, requests and the like such that a government compliant record may be maintained for each operation or output generated by the system 400.

The cloud-based anatomical data processing system 400 may also include a payment system 316 coupled to a cloud-based payment component 410. In the current example, the payment system 316 may be a payment processing application or equipment in proximity to the patient and/or healthcare professional for issuing payment for the requested jobs. A data warehouse 308 may be coupled to the payment component 410 for storing the payment data.

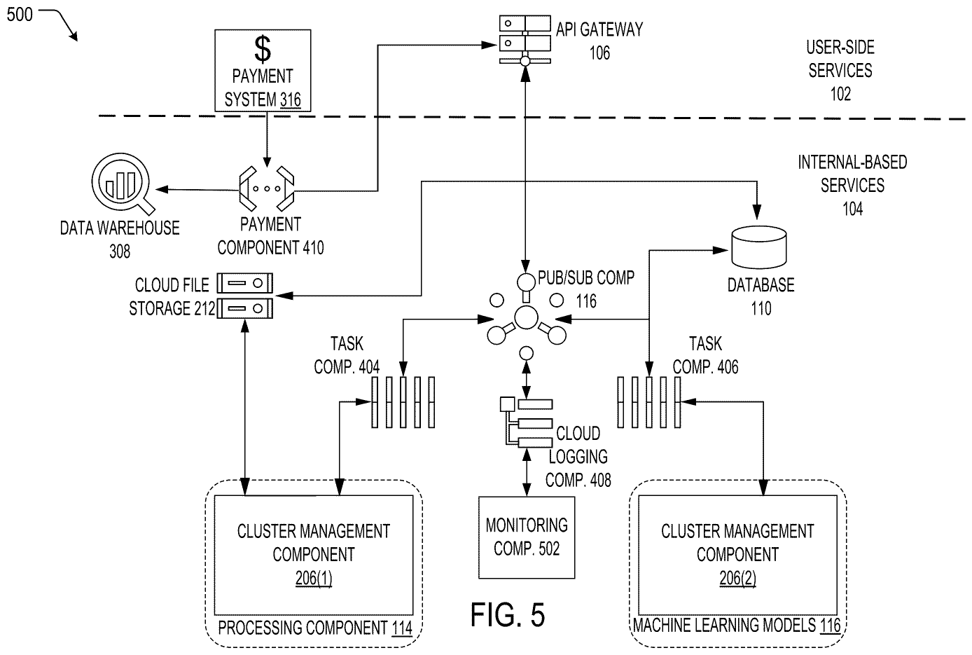

FIG. 5 is another example block diagram of an architecture for a cloud-based anatomical data processing system 500 according to some implementations. In the current example, the API gateway 106 may be in communication with the publish-subscribe component 112, the database 110, and the cloud file storage 212, discussed above with respect to FIGS. 1 and 2. The publish-subscribe component 112 may be further coupled to a task component 404 associated with a first cluster management component 206(1) and a task component 406 associated with a second cluster management component 206(2).

Each of the cluster management components 206(1) and 206(2) are, respectively, associated with the operations of the processing component 114 and the machine learning models 116. Accordingly, in the current implementation, the processing component 114 is associated with the task component 404 and the cluster management component 206(1) and the machine learning models 116 are associated with the task component 406 and the cluster management component 206(2). In this manner, the operations, scheduling, and/or processing associated with determining metrics, measurement, landmarks and the like may be separated by the system 400 from the operations, scheduling, and/or processing associated with diagnostics, identifying symptoms and conditions, and generating recommended or patient customized therapy and/or treatments.

The publish-subscribe component 112 may also be coupled to a cloud logging component 408. The cloud logging component 408 may be configured to record events, jobs, tasks, requests and the like such that a government compliant record may be maintained for each operation or output generated by the system 400. For example, the cloud logging component 408 may receive the log data from a monitoring component 502 that may monitor the operations, scheduling, and/or processing associated with the processing component 114 and/or the machine learning models 116. In this manner, the cloud logging component 408 and the monitoring component 502 may be utilized to provide events for trouble shooting the cloud-based system 500, and to provide governmental compliance data based on the various jurisdictions associated with the patients and/or healthcare professionals.

The cloud-based anatomical data processing system 400 may also include a payment system 316 coupled to a cloud-based payment component 410. In the current example, the payment system 316 may be a payment processing application or equipment in proximity to the patient and/or healthcare professional for issuing payment for the requested jobs. A data warehouse 308 may be coupled to the payment component 410 for storing the payment data.

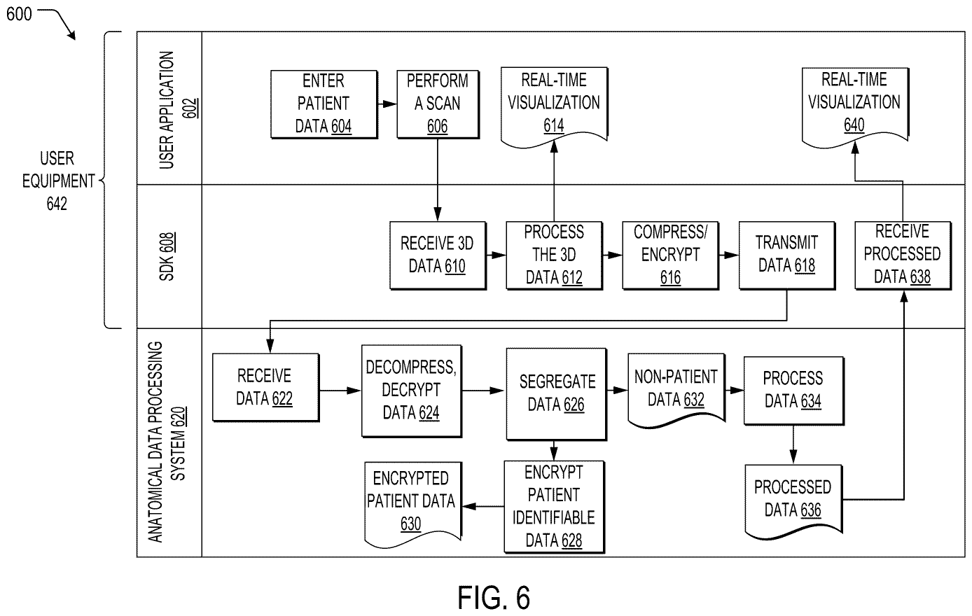

FIG. 6 is an example process flow diagram 600 associated with the cloud-based anatomical data processing system of FIGS. 1-5 described according to some implementations. In the current example, the user equipment 642 may be utilized by a healthcare professional to enter patient data 604 and subsequently perform a scan 606 of the patient to capture the 3D data that is provided to the cloud-based system 620. For example, the user (e.g., healthcare professional) may utilize an application 602 hosted by the user equipment 602 to enter patient data 604 and initiate a scanning session 606 of the patient to capture both the meta data (e.g., the patient data) and the 3D data. An SDK 608, for instance, installed on the user equipment 642, may receive the 3D data 610 being generated via the scan performed 606. The SDK 608 may process the 3D data 612. For example, the SDK 608 may generate TSDF data, mesh data, and/or convert the 3D data into one or more types of files for output.

The processed data may then be displayed as a substantially real-time visualization 614 via the application 602 hosted on the user equipment 642, as illustrated. In this manner, concurrent feedback may be provided to the patient and/or healthcare professional performing the scan via the user equipment 642. In some cases, the user application 602 and/or the SDK 604 may perform SLAM tracking of the scan to determine a quality, completion, and/or holes within the 3D data. The tracking data may then be provided as part of the substantially real-time visualization 614 so that the patient and/or healthcare professional may update the scan while still capturing the 3D data.

The processed data may also be compressed and/or encrypted 616 by the SDK 608 and the data may then be transmitted 618 to the anatomical data processing system 620, as discussed herein. The anatomical data processing system 620 may receive the data (e.g., the compressed, encrypted 3D data and associated meta data) 622 and decompress and decrypt the data 624.

The anatomical data processing system 620 may also segregate the data 626 into patient identifiable data 630 and non-patient data 632. The anatomical data processing system 620 may generate keys and encrypt the patient identifiable data 628 which is then stored as encrypted patient data 630. The anatomical data processing system 620 may then provide the encrypted patient data 630 to various system such as a datastore associated with the health care provider associated with the scan. The anatomical data processing system 620 may process the non-patient data 632 to perform various diagnostics assistance features. For instance, the anatomical data processing system 620 may determine, classify, or identify body-parts, landmarks, anthropometric metrics or features, potential conditions or symptoms, recommend treatments, or the like. In various instances, the additional processing 634 may be performed on-demand and/or at the request of the user (e.g., the healthcare professional). For instance, the user may select the processing to be performed by the cloud-based system via the SDK 608 or downloadable application on the user equipment 602 prior to or in conjunction with transmitting the 3D data to the anatomical data processing system 620. In this manner, the user may limit the processing performed by the anatomical data processing system 620 based at least in part on the services being provided to the patient.

The processed data 636 may then be transmitted back to the SDK 608. For instance, the processed data 636 may be again encrypted and compressed and received by the SDK 638. The SDK 608 may then update, replace, or generate a second real-time visualization 640 of the original 3D data captured as part of the scan. The real-time visualization 640 may also be displayed by the user equipment 602 to the user (e.g., the healthcare professional and/or the user).

In the current example, the SDK 604 is shown as on the user equipment 642. However, it should be understood, that the SDK 604 may be hosted in part on the user equipment 642 and partly in the cloud as part of the system 620. In other cases, the SDK 604 may be completely hosted by the cloud-based system 620 such that only the application 602 is on the local user equipment 642.

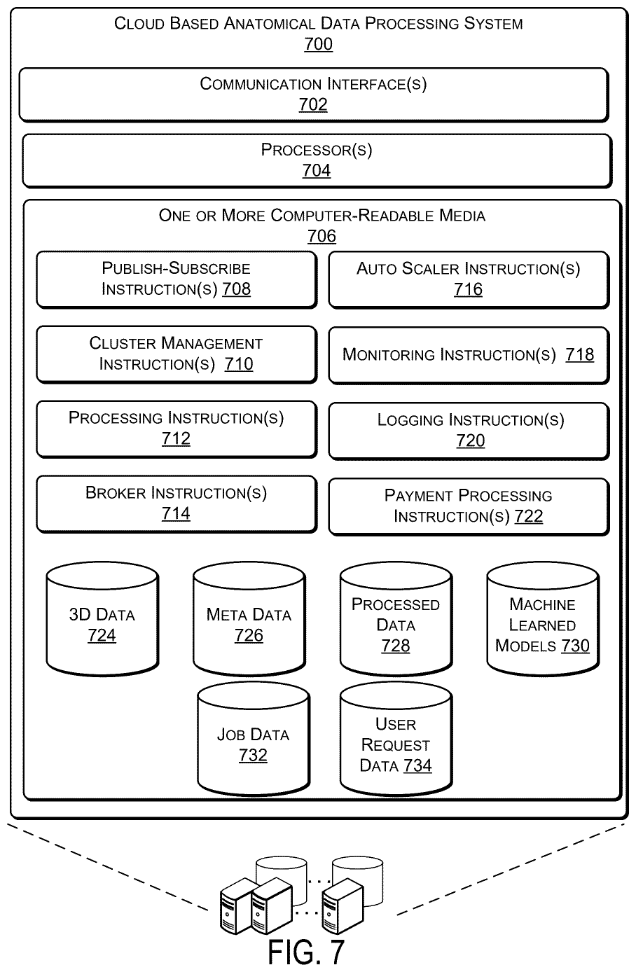

FIG. 7 is an example cloud-based anatomical data processing system 700 that may implement the techniques described herein according to some implementations. The cloud-based anatomical data processing system 700 can include one or more communication interface(s) 702 that enables communication between the cloud-based anatomical data processing system 700 and one or more other local or remote computing device(s) or remote services, such as the user equipment. For instance, the communication interface(s) 702 can facilitate communication with other proximate sensor systems and/or other facility systems. The communications interfaces(s) 702 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The cloud-based anatomical data processing system 700 may include one or more processors 704 and one or more computer-readable media 706. Each of the processors 704 may itself comprise one or more processors or processing cores. The computer-readable media 706 is illustrated as including memory/storage. The computer-readable media 706 may include volatile media (such as random access memory (RAM)) and/or nonvolatile media (such as read only memory (ROM), Flash memory, optical disks, magnetic disks, and so forth). The computer-readable media 706 may include fixed media (e.g., RAM, ROM, a fixed hard drive, and so on) as well as removable media (e.g., Flash memory, a removable hard drive, an optical disc, and so forth). The computer-readable media 706 may be configured in a variety of other ways as further described below.

Several modules such as instructions, data stores, and so forth may be stored within the computer-readable media 706 and configured to execute on the processors 704. For example, as illustrated, the computer-readable media 706 stores publish-subscribe instructions 708, cluster management instructions 710, processing instructions 712, broker instructions 714, auto scaler instructions 716, monitoring instruction 718, logging instruction 720, payment processing instructions 722, discussed above, as well as other instructions, such as an operating system. The computer-readable media 706 may also be configured to store data, such as 3D data 724, meta data 726, processed data 728 (e.g., mesh data, metric data, measurement data, diagnostic results, landmark data, therapy data, and the like), machine learned models 730, job data 732, user request data 734 as well as other data.

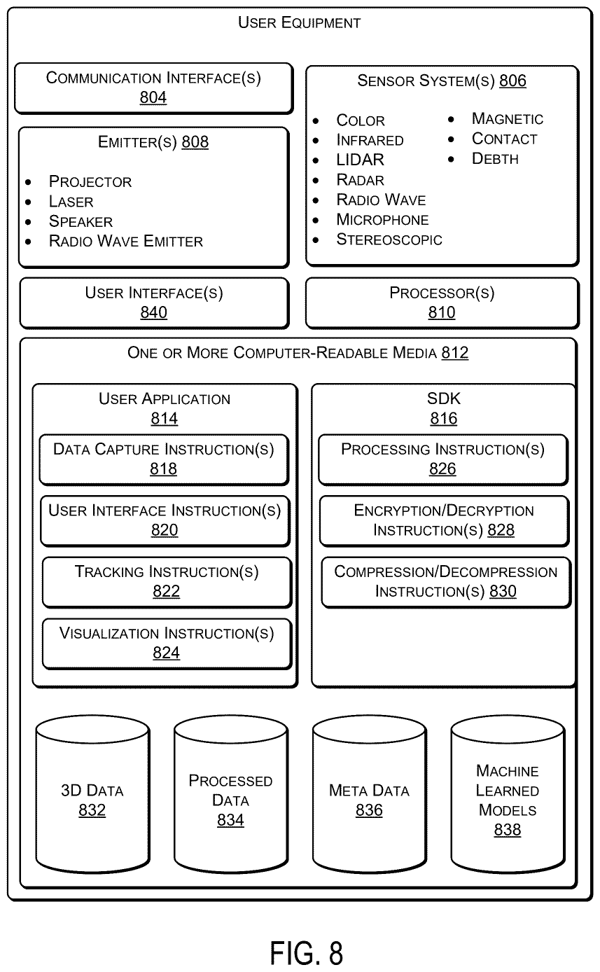

FIG. 8 is an example user equipment 800 that may implement the techniques described herein according to some implementations. The image device 800 may include one or more communication interface(s) 804 (also referred to as communication devices and/or modems), one or more sensor system(s) 806, and one or more emitter(s) 808.

The user equipment 800 can include one or more communication interfaces(s) 804 that enable communication between the user equipment 800 and one or more other local or remote computing device(s) or remote services, such as a cloud-based system of FIGS. 1-5. For instance, the communication interface(s) 804 can facilitate communication with other proximate sensor systems, a central control system, or other facility systems. The communications interfaces(s) 804 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The one or more sensor system(s) 806 may be configured to capture the 3D data 832 (or other image based data) associated with a patient. In at least some examples, the sensor system(s) 806 may include thermal sensors, time-of-flight sensors, location sensors, LIDAR sensors, radar sensors, sonar sensors, infrared sensors, cameras (e.g., RGB, IR, intensity, depth, etc.), magnetic sensors, microphone sensors, environmental sensors (e.g., temperature sensors, humidity sensors, light sensors, pressure sensors, etc.), and the like. In some examples, the sensor system(s) 806 may include multiple instances of each type of sensor. For instance, camera sensors may include multiple cameras disposed at various locations.

The user equipment 800 may also include one or more emitter(s) 808 for emitting light and/or sound. By way of example and not limitation, the emitters in this example include light, illuminators, lasers, patterns, such as an array of light, audio emitters, and the like.

The user equipment 800 may also include one or more user interfaces 840, such as input (e.g., a display) or output devices (e.g., mouse or keyboard). The user interfaces 840 may include a virtual environment display or a traditional two-dimensional display, such as a liquid crystal display or a light emitting diode display. The user interfaces 840 may also include one or more input components for receiving feedback from the user. In some cases, the input components may include tactile input components, audio input components, or other natural language processing components. In one specific example, the user interfaces 840 may be a combined touch enabled display.

The user equipment 800 may include one or more processors 810 and one or more computer-readable media 812. Each of the processors 810 may itself comprise one or more processors or processing cores. The computer-readable media 812 is illustrated as including memory/storage. The computer-readable media 812 may include volatile media (such as random access memory (RAM)) and/or nonvolatile media (such as read only memory (ROM), Flash memory, optical disks, magnetic disks, and so forth). The computer-readable media 812 may include fixed media (e.g., RAM, ROM, a fixed hard drive, and so on) as well as removable media (e.g., Flash memory, a removable hard drive, an optical disc, and so forth). The computer-readable media 812 may be configured in a variety of other ways as further described below.

Several modules such as instructions, data stores, and so forth may be stored within the computer-readable media 812 and configured to execute on the processors 810. For example, as illustrated, the computer-readable media 812 stores a user application 814 and an SDK 816 as discussed above. The user application 814 may include data capture instructions 818 (e.g., scanning instructions), user interface instructions 820 (e.g., for receiving inputs and outputting data such as visualization), tracking instructions 822 (e.g., SLAM operations), visualization instructions 824. The SDK 816 may include processing instructions 826 (e.g., TSDF or mesh generation instructions), encryption and decryption instructions 828, and compression and decompression instructions 830. The computer-readable media 812 may also be configured to store data, such as 3D data 832, processed data 834, meta data 836, machine learned models 838, and the like.

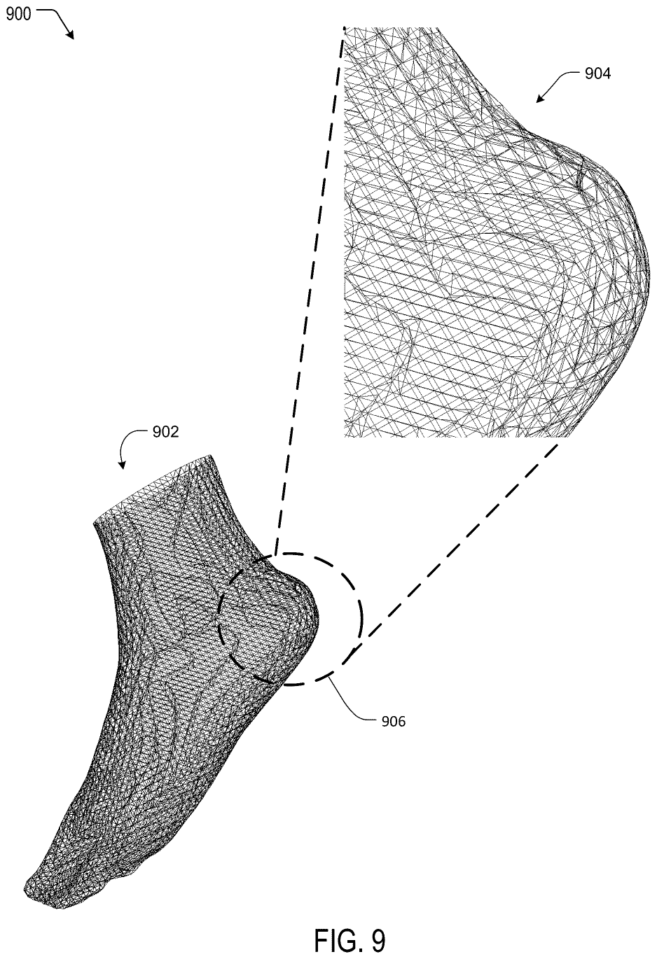

FIG. 9 is an example pictorial diagram that shows 3D data 900 of a foot of a patient according to some implementations. As illustrated, the 3D data 900 is shown in a first view 902 of the full foot and a zoomed in view 904 of the heel 906. In this example, the 3D data is in a mesh form, such as generated by the SDK and/or cloud-based system discussed above.

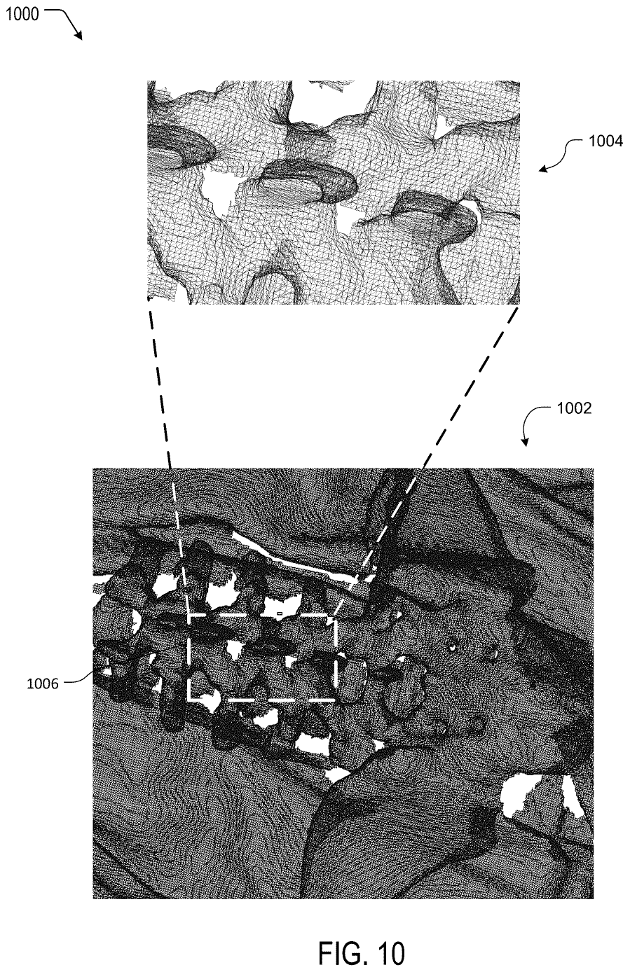

FIG. 10 is another example pictorial diagram that shows 3D data 1000 of a spine of a patient according to some implementations. As illustrated, the 3D data 1000 is shown in a first view 1002 and a zoomed in view 1004 of an area 1006 of the spine. In this example, the 3D data is again in a mesh form, such as generated by the SDK and/or cloud-based system discussed above.

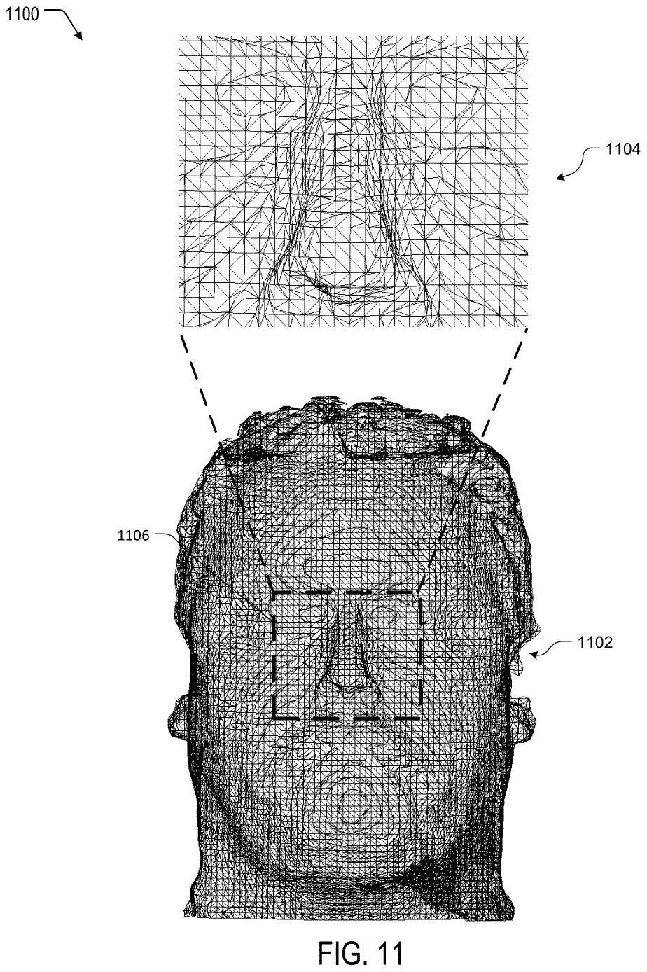

FIG. 11 is another example pictorial diagram that shows 3D data 1100 of a face of a patient according to some implementations. As illustrated, the 3D data 1100 is shown in a first view 1102 and a zoomed in view 1104 of an area 1106 of the face, such as the nose region. In this example, the 3D data is again in a mesh form, such as generated by the SDK and/or cloud-based system discussed above.

Although the discussion above sets forth example implementations of the described techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

### EXAMPLE CLAUSES

A. A system comprising: an application programming interface (API) gateway in wireless communication with a user equipment; a cluster management component coupled to the API gateway, the cluster management component to perform orchestration and scaling operations on three-dimensional data of one or more features of a patient; a processing component coupled to the cluster management component to perform operations on the three-dimensional data of the one or more features of the patient; one or more machine learning models coupled to the cluster management component to perform operations on the three-dimensional data of the one or more features of the patient, the one or more machine learning models trained on image data associated with patients having various conditions, symptoms, cultural backgrounds, ages, genders, and physical characteristics together with symptom and treatment data associated with various ailments; and a database coupled to the cluster management component for storing data associated with the system, the data including the three-dimensional data.
B. The system of claim A, wherein the user equipment is a medical scanning device and the API gateway is configured to receive the three-dimensional data of the patient, meta data associated with the patient, and a healthcare professional request from the user equipment, the healthcare professional request including an operation to be performed by the system.
C. The system of claim B, wherein the operations performed on the three-dimensional data is at least one of the following: a landmark detection operation; a metric determining operation; a measurement determining operation; a diagnostic operation; an operation to determine a personalized therapy or treatment for the patient; or an operation to determine a symptom or condition of the patient.
D. The system of claim C, wherein: the processing component is configured to perform the landmark detection operation, the metric determining operation, and the measurement determining operation; and the machine learning models are configured to perform the diagnostic operation, the operation to determine the personalized therapy or treatment for the patient, and the operation to determine the symptom or condition of the patient.
E. The system of claim A, further comprising: a publish-subscribe component coupled to the cluster management component, the publish-subscribe component to set quotas and rate limits for healthcare professional requests by different users and prioritize incoming tasks; and a broker component coupled to the publish-subscribe component, the broker component to manage messages within the system.
F. The system of claim E, further comprising: a cloud logging component coupled to the publish-subscribe component, the cloud logging component to generate log data associated with operations of the processing component and the one or more machine learned models.
G. The system of claim E, further comprising: a first task component coupled to the publish-subscribe component, the first task component to manage the operations of the processing component; and a second task component coupled to the publish-subscribe component, the second task component to manage the operations of the one or more machine learning models.
H. The system of claim A, wherein the database includes a first portion for storing personalized identifiable data associated with the patient and a second portion segmented from the first portion for storing non-personalized identifiable data.
I. A method comprising: capturing, via an application hosted on a user equipment, patient data and three-dimensional data of a feature of a patient; processing, concurrently with the capturing and via a software development kit (SDK) associated with the user equipment, the three-dimensional data to generate a first mesh; presenting, concurrently with the capturing and via the application and the user equipment, the first mesh as a first visualization; transmitting the three-dimensional data and the patient data to an anatomical data processing system; performing operations on the three-dimensional data to generate processed data associated with the patient; transmitting the processed data to the SDK; and presenting, via the application and the user equipment, a second visualization on the user equipment, the second visualization representing the feature of the patient.
J. The method of claim I, wherein the first visualization includes at least one indicator, the at least one indicator to provide a user with a visual indication to guide the user in capturing additional three-dimensional data.
K. The method of claim I, further comprising: compressing the three-dimensional data prior to transmitting to the anatomical data processing system; and encrypting the three-dimensional data prior to transmitting to the anatomical data processing system.
L. The method of claim I, further comprising segregation, by the anatomical data processing system, the patient data into patient identifiable data and non-patient identifiable data.
M. The method of claim I, wherein performing the operations on the three-dimensional data to generate processed data associated with the patient further comprises determining at least one of a landmark, a metric, or a measurement associated with the feature of the patient.
N. The method of claim I, wherein performing the operations on the three-dimensional data to generate processed data associated with the patient further comprises inputting the three-dimensional data into one or more machine learning models trained on image data associated with patients having various conditions, symptoms, cultural backgrounds, ages, genders, and physical characteristics together with symptom and treatment data associated with various ailments, and receiving as an output of a diagnostic result, a personalized therapy or treatment for the patient, a symptom, or condition of the patient.
O. The method of claim I, further comprising: compressing the processed data prior to transmitting to the SDK; and encrypting the processed data prior to transmitting to the SDK.
P. A system comprising: an application programming interface (API) gateway to receive three-dimensional data of a feature of a patient from user equipment; a cluster management component coupled to the API gateway, the cluster management component comprising: a processing component coupled to perform operations on the three-dimensional data of the feature of the patient; one or more machine learning models coupled to the cluster management component to perform operations on the three-dimensional data of the feature of the patient, the one or more machine learning models trained on image data associated with patients having various conditions, symptoms, cultural backgrounds, ages, genders, and physical characteristics together with symptom and treatment data associated with various ailments; a first auto-scaler coupled to the processing component; and a second auto-scaler coupled to the one or more machine learning models; and a database coupled to the cluster management component for storing data associated with the system, the data including the three-dimensional data.
Q. The system of claim P, further comprising a broker component coupled to the cluster management component, the broker component to manage messages within the system and further comprising: a publish-subscribe component to set quotas and rate limits for healthcare professional requests by different users and prioritize incoming tasks; and a cloud file storage.
R. The system of claim P, wherein the operations performed on the three-dimensional data is at least one of the following: a landmark detection operation; a metric determining operation; a measurement determining operation; a diagnostic operation; an operation to determine a personalized therapy or treatment for the patient; or an operation to determine a symptom or condition of the patient.
S. The system of claim R, wherein the processing component is configured to perform the landmark detection operation, the metric determining operation, and the measurement determining operation.
T. The system of claim R, wherein the machine learning models are configured to perform the diagnostic operation, the operation to determine the personalized therapy or treatment for the patient, and the operation to determine the symptom or condition of the patient.

While the example clauses described above are described with respect to one particular implementation, it should be understood that, in the context of this document, the content of the example clauses can also be implemented via a method, device, system, a computer-readable medium, and/or another implementation. Additionally, any of examples A-T may be implemented alone or in combination with any other one or more of the examples A-T.

### CONCLUSION

While one or more examples of the techniques described herein have been described, various alterations, additions, permutations and equivalents thereof are included within the scope of the techniques described herein. As can be understood, the components discussed herein are described as divided for illustrative purposes. However, the operations performed by the various components can be combined or performed in any other component. It should also be understood that components or steps discussed with respect to one example or implementation may be used in conjunction with components or steps of other examples.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

1. A system comprising:
an application programming interface (API) gateway in wireless communication with a user equipment;
a cluster management component coupled to the API gateway, the cluster management component to perform orchestration and scaling operations on three-dimensional data of one or more features of a patient;
a processing component coupled to the cluster management component to perform operations on the three-dimensional data of the one or more features of the patient;
one or more machine learning models coupled to the cluster management component to perform operations on the three-dimensional data of the one or more features of the patient, the one or more machine learning models trained on image data associated with patients having various conditions, symptoms, cultural backgrounds, ages, genders, and physical characteristics together with symptom and treatment data associated with various ailments; and
a database coupled to the cluster management component for storing data associated with the system, the data including the three-dimensional data.

2. The system of claim 1, wherein the user equipment is a medical scanning device and the API gateway is configured to receive the three-dimensional data of the patient, meta data associated with the patient, and a healthcare professional request from the user equipment, the healthcare professional request including an operation to be performed by the system.

3. The system of claim 1, wherein the operations performed on the three-dimensional data is at least one of the following:
a landmark detection operation;
a metric determining operation;
a measurement determining operation;
a diagnostic operation;
an operation to determine a personalized therapy or treatment for the patient; or
an operation to determine a symptom or condition of the patient.

4. The system of claim 1, wherein:
the processing component is configured to perform the landmark detection operation, the metric determining operation, and the measurement determining operation; and
the machine learning models are configured to perform the diagnostic operation, the operation to determine the personalized therapy or treatment for the patient, and the operation to determine the symptom or condition of the patient.

5. The system of claim 1, further comprising:
a publish-subscribe component coupled to the cluster management component, the publish-subscribe component to set quotas and rate limits for healthcare professional requests by different users and prioritize incoming tasks; and
a broker component coupled to the publish-subscribe component, the broker component to manage messages within the system.

6. The system of claim 5, further comprising:
a cloud logging component coupled to the publish-subscribe component, the cloud logging component to generate log data associated with operations of the processing component and the one or more machine learned models;
a first task component coupled to the publish-subscribe component, the first task component to manage the operations of the processing component; and
a second task component coupled to the publish-subscribe component, the second task component to manage the operations of the one or more machine learning models.

7. The system of claim 1, wherein the database includes a first portion for storing personalized identifiable data associated with the patient and a second portion segmented from the first portion for storing non-personalized identifiable data.

8. A method comprising:
capturing, via an application hosted on a user equipment, patient data and three-dimensional data of a feature of a patient;
processing, concurrently with the capturing and via a software development kit (SDK) associated with the user equipment, the three-dimensional data to generate a first mesh;
presenting, concurrently with the capturing and via the application and the user equipment, the first mesh as a first visualization;
transmitting the three-dimensional data and the patient data to an anatomical data processing system;
performing operations on the three-dimensional data to generate processed data associated with the patient;
transmitting the processed data to the SDK; and
presenting, via the application and the user equipment, a second visualization on the user equipment, the second visualization representing the feature of the patient.

9. The method of claim 8, wherein the first visualization includes at least one indicator, the at least one indicator to provide a user with a visual indication to guide the user in capturing additional three-dimensional data.

10. The method of claim 8, further comprising:
compressing the three-dimensional data prior to transmitting to the anatomical data processing system; and
encrypting the three-dimensional data prior to transmitting to the anatomical data processing system.

11. The method of claim 8, further comprising segregating, by the anatomical data processing system, the patient data into patient identifiable data and non-patient identifiable data.

12. The method of claim 8, wherein performing the operations on the three-dimensional data to generate processed data associated with the patient further comprises determining at least one of a landmark, a metric, or a measurement associated with the feature of the patient.

13. The method of claim 8, wherein performing the operations on the three-dimensional data to generate processed data associated with the patient further comprises inputting the three-dimensional data into one or more machine learning models trained on image data associated with patients having various conditions, symptoms, cultural backgrounds, ages, genders, and physical characteristics together with symptom and treatment data associated with various ailments, and receiving as an output of a diagnostic result, a personalized therapy or treatment for the patient, a symptom, or condition of the patient.

14. (canceled)

15. (canceled)

16. One or more non-transitory computer-readable media storing instructions that, when executed by one or more processors, cause the one or more processors to:
capture, via an application hosted on a user equipment, patient data and three-dimensional data of a feature of a patient;
process, concurrently with the capturing and via a software development kit (SDK) associated with the user equipment, the three-dimensional data to generate a first mesh;
present, concurrently with the capturing and via the application and the user equipment, the first mesh as a first visualization;
transmit the three-dimensional data and the patient data to an anatomical data processing system;
perform operations on the three-dimensional data to generate processed data associated with the patient;
transmit the processed data to the SDK; and
present, via the application and the user equipment, a second visualization on the user equipment, the second visualization representing the feature of the patient.

17. The one or more non-transitory computer-readable media of claim 16, wherein the first visualization includes at least one indicator, the at least one indicator to provide a user with a visual indication to guide the user in capturing additional three-dimensional data.

18. The one or more non-transitory computer-readable media of claim 16, wherein the instructions when executed by the one or more processors, cause the one or more processors to:
compress the three-dimensional data prior to transmitting to the anatomical data processing system; and
encrypt the three-dimensional data prior to transmitting to the anatomical data processing system.

19. The one or more non-transitory computer-readable media of claim 16, wherein the instructions when executed by the one or more processors, cause the one or more processors to segregate the patient data into patient identifiable data and non-patient identifiable data.

20. The one or more non-transitory computer-readable media of claim 16, wherein performing the operations on the three-dimensional data to generate processed data associated with the patient further comprises determining at least one of a landmark, a metric, or a measurement associated with the feature of the patient.

21. The one or more non-transitory computer-readable media of claim 16, wherein performing the operations on the three-dimensional data to generate processed data associated with the patient further comprises inputting the three-dimensional data into one or more machine learning models trained on image data associated with patients having various conditions, symptoms, cultural backgrounds, ages, genders, and physical characteristics together with symptom and treatment data associated with various ailments, and receiving as an output of a diagnostic result, a personalized therapy or treatment for the patient, a symptom, or condition of the patient.

22. The one or more non-transitory computer-readable media of claim 16, wherein the instructions when executed by the one or more processors, cause the one or more processors to:
compress the processed data prior to transmitting to the SDK; and
encrypt the processed data prior to transmitting to the SDK.
